(19) United States
(12) Patent Application Publication
(10) Pub. No.: US 2017/0191086 A1
(43) Pub. Date: Jul. 6, 2017

(54) PROCESS FOR THE TRANSFORMATION OF ESSENTIAL OILS COMPRISING LINALOL

(71) Applicant: Natura Cosméticos S.A., Sao Paulo (BR)

(72) Inventors: Renata BUELONI, Sao Paulo (BR); Renata Rabelo SCHEFER, Sao Paulo (BR); Roberta ROESLER, Sao Paulo (BR)

(21) Appl. No.: 14/984,607

(22) Filed: Dec. 30, 2015

(51) Int. Cl. C12P 7/04; C11B 9/00

(52) U.S. Cl. CPC C12P 7/04; C11B 9/0015

(57) ABSTRACT

A process for transforming essential oils comprising linalool and the production of mixtures of linalool derivatives useful as fragrances in cosmetic compositions.

### FIELD OF THE INVENTION

The present invention relates to a process of transforming essential oils comprising linalool, and to the production of mixtures of linalool derivatives useful as fragrances in cosmetic compositions.

### BACKGROUND OF THE INVENTION

Terpenes are a large and diverse class or organic compounds produced by a variety of plants, as for example conifers (Firn, Richard (2010). Nature's Chemicals. Oxford: Biology)). Terpenoids are related compounds that contain additional functional groups.

Terpenes and terpenoids are the main constituents of essential oils of many types of plants and flowers. Essential oils are widely used as fragrances in perfumery. Variations and synthetic derivatives of natural terpenes and terpenoids expand widely the variety of aromas used in perfumery and aromas used in food additives.

Plant terpenoids are extensively used for their aromatic qualities, contributing, for instance, to the eucalyptus aroma, the cinnamon, clove and ginger flavors, and the yellow color in sunflowers and the red color in tomatoes. Well known terpenoids include citral, mentol, camphor, ginkgolide and bilobalide found in Ginkgo biloba and the curcuminoids found in saffron and mustard seed.

Linalool is a natural terpene alcohol found in many flowers and spice plants with many commercial applications, most of which are based on its pleasant aroma (floral, with a spice touch). It has other names such as β-linalool, linalyl alcohol, linaloyl oxide, p-linalool, alo-ocimenol, and 3,7-dimethyl-1,6-octadien-3-ol.

The use of linalool derivatives such as linalool oxides as fragrances ion perfumery compositions is already known from the prior art. WO2011101182 teaches a perfume composition comprising fenchone. WO2015042232 and WO2008145582 teach fragrant compositions comprising fenchol.

Besides, Miranda et al (Linalool biotransformation with fungi, Expression of Multidisciplinary Flavour Science, pp 349-353), teaches the obtainment of linalool oxides by biotransformation with fungi and the use thereof as flavoring agents.

Document U.S. Pat. No. 6,703,218 further teaches a process for preparing mixtures comprising linalool oxides through the action of a lipase of microorganisms in the presence of hydrogen peroxide, as well as the aromatic property of said compounds.

However, there is a constant demand for new fragrances on the perfumery market.

The present invention teaches a surprising process by which new mixtures of linalool derivatives can be produced from vegetable essential oils comprising linalool so as to obtain new fragrances.

### SUMMARY OF THE INVENTION

The present invention teaches a process for transforming essential oils comprising linalool, by which one produces mixtures of linalool derivatives useful as fragrances in cosmetic compositions. The method of the present invention comprises transforming essential oils by means of microorganisms expressing the cytochrome P450 protein.

As a result, the present invention further provides mixtures of linalool derivatives obtained by the process according to the present invention, useful for the preparation of cosmetic compositions, as well as cosmetic compositions comprising said mixtures.

### DETAILED DESCRIPTION OF THE PRESENT INVENTION

Linalool is a natural terpene alcohol found in many flowers and spice plants, known also as β-linalool, linalyl alcohol, linaloyl oxide, p-linalool, alo-ocimenol, and 3,7-dimethyl-1,6-octadien-3-ol. More than 200 species of plants produce linalool, chiefly those of the Laminaceae family (mint, aromatic herbs), Lauraceae family (laurel tree, cinnamon, rose-wood), and Rutaceae family (citric fruits), Birch, cannabis and other plants from tropical to boreal climatic zones, as well as a few fungi.

An essential oil is a hydrophobic liquid concentrate containing volatile compounds of plant aroma. Essential oils are also known as volatile oils, ethereal oils or simply as oil from the plant from which they have been extracted, such as clove oil. An oil is “essential” in the sense that it contains the “essence” of the plant fragrance—the characteristic fragrance of the plant from which it derives. The term “essential” as used herein does not mean indispensable, as in the case of essential amino acids or essential fatty acids, which are thus called because they are nutritionally demanded by a determined live organism.

Essential oils are generally extracted by distillation, often by using vapor. Other processes include expression, extraction with solvent, extraction of absolute petroleum, resining and cold pressing. They are used in perfumes, cosmetics, toilet-soaps and other products, for flavoring foods and beverages, and for imparting aromas to incense and household cleaning products.

Linalool is a natural terpene alcohol found in many flowers and spice plants with many commercial applications, most of which are based on its pleasant aroma (floral, with a spice touch). It has other names such as β-linalool, linalyl alcohol, linaloyl oxide, p-linalool, alo-ocimenol, and 3,7-dimethyl-1,6-octadien-3-ol.

A few examples of essential oils comprising linalool that can be transformed according to the present invention are essential oils of plants of the genera Ocimum, Copaifera, Mentha, and Cyperus. Preferably, essential oils are of hoary basil (Ocimum americanum), copaiba (Copaifera officinalis L., C. guianensis, C. reticulate, C. multijuga, C. confertiflora, C. langsdorffii, C. cariacea and C. cearensis), pudding grass (Mentha pulegium) and priprioca (Cyperus articulatus).

According to the present invention, the essential oil comprising linalool is transformed by reaction with a microorganism expressing a P450 cytochrome protein, producing a mixture of linalool derivatives. Preferably, the linalool derivatives are linalool oxides.

The microorganism expressing a P450 cytochrome protein is a transgenic microorganism. More preferably, the transgenic microorganism is yeast. More preferably, the yeast is Sccharomyces cerevisiae.

The P45 cytochrome protein expressed in the transgenic microorganism used in the process of the present invention is preferably a farneseno synthase. More preferably, the P450 cytochrom protein is the farneseno synthase of Artemisia annua L., CYP71AV1. A region encoding farneseno synthase of Artemisia annua L., CYP71AV1 useful in the present invention can be obtained, for instance, from the access number GenBank No. AY835398.

Examples of transgenic microorganisms expressing a P45 cytochrome protein of the farneseno synthase type, as well as its amino acid sequences and encoding sequences and genic constructions useful in the present invention can be found in documents: WO2012106405, WO200614837, WO200705604, WO200839499, WO2012149470, WO201595804, WO2012158466, US7172886, WO200685899, WO200914636, WO2008140492, WO2008133658, WO2009126623, WO2007139924, WO2007136847, WO200942070, WO201371172, WO200727338 and WO2012135591, incorporated herein by reference.

The process of transforming essential oils comprising linalool of the present invention can be further carried out with yeasts stains available commercially as, for example, the stains Y1979 and Y5056 (Amyris Brasil S.A.).

The essential oil to be transformed is added to the culture medium of the transgenic microorganism and then incubated, preferably according to the parameters below.

The biotransformation reaction for transforming essential oil according to the present invention is preferably carried out in an aerobic medium. Besides, the culture medium is kept preferably under stirring during the reaction.

Preferably, the cell density of the microorganism ranges from 1.5 to about 20. Optionally, the cell density is of about 1.5, 2, 3, 4, 5, 6, 7, 8, 9, 10, 11, 12, 13, 14, 15, 16, 17, 18, 19 or 20. More preferably, the cell density is of about DO=10.

Preferably, the reaction time if of about 30, 31, 32, 33, 34, 35, 36, 37, 38, 39, 40, 41, 42, 43, 44, 45, 46, 47, 48, 49 or 50 hours. More preferably, the reaction time is of about 30 and about 40 hours.

Preferably, the reaction temperature ranges from about 24 to about 37° C. Optionally, the temperature is of about 24, 25, 26, 27, 28, 29, 30, 31, 32, 33, 34, 35, 36 or 37 ° C. More preferably, the temperature ranges from about 24 to 30 ° C. Still more preferably it is of about 30 ° C.

Preferably, the pH of the reaction is between about pH 4.0 and pH 7.0. Optionally, the pH of the reaction is of about pH 4.0 4.5, 5.0, 5.5, 6.0, 6, or 7.0. More preferably the pH of the reaction is of about pH 7.0.

The present invention further provides cosmetic compositions comprising the linalool derivatives obtained according to the process described herein.

### EXAMPLE 1

In reaction tubes of the falcon and erlenmaeyer type, one added volumes of 1, 2, 3, 4 and 5 mL of culture medium (DO=1.5, 3, 10 and 20) of Saccharomyces cerevisiae expressing the P450 cytochrome protein and of hoary basil (Ocimum americanum) essential oil. Reaction times of 37-40 and 48-50 hours, raction times of 24, 30 and 37 ° C. and pH de 4.0, 5.5 and 7.0 were tested

In the mixture of linalool derivatives obtained under the above conditions, one detected cis furanoid linalool oxide, cis pyranoid linalool oxide, epoxylinalool, 5-hydroxylinalool, 8-hydroxylinalool, hotrienol, fenchone, fenchol, limonene oxide, among others.

The results referring to the obtainment of the above compounds are represented in Table 1.

TABLE 1

| Parameter | Results |
|---|---|
| Reaction tube | Falcon proved to be a superior option to provide |
| Falcon x Erlenmeyer | ideal aeration for 2 mL culture and to reduce |
| tube | loss of substrate and product by evaporation |
| Cell density | 10 OD-mL ensured the highest amount of P450 |
| OD-ml = 1, 5, 3, 10 | present, with less loss of material in biomass |
| and 20 | |
| Culture Volume: | 1 and 2 mL performed in a similar way or |
| 1, 2, 3, 4, 5 mL (to 10 | better than the others. |
| OD-mL, proportion- | |
| ally stepped substrate) | |
| Reaction time: | More incubation time led to a few low-intensity |
| 37-40 h vs 48-50 h | peaks. |
| Reaction temperature: | Conversion tendency: 24° C. > 30° C. > 37° C. |
| 24, 30, 37° C. | |
| pH of the reaction | 7.0 > 5.5 > 4.0 |
| 4.0, 5.5, 7.0 | |

1. A process for transforming essential oils comprising linalool, which comprises incubating an essential oil comprising linalool in a culture medium of microorganism that expresses a P450 protein.

2. The process according to claim 1, wherein the essential oil is from a plant belonging to the genus selected from the group consisting of Ocimum, Copaifera, Mentha, and Cyperus.

3. The process according to claim 1, wherein the essential oil is from a plant selected from the group consisting hoary basil (Ocimum americanum), copaiba (Copaifera officinalis L., C. guianensis, C. reticulate, C. multijuga, C. confertiflora, C. langsdorffii, C. cariacea and C. cearensis), pudding grass (Mentha pulegium) and priprioca (Cyperus articulatus).

4. The process according to 1, wherein the microorganism is transgenic yeast.

5. The process according to claim 4, wherein the P450 cytochrome protein is farneseno synthase of Artemisia annua L., CYP71AV1.

6. The process according to claim 1, wherein the reaction is carried out in an aerobic medium.

7. The process according to claim 1, wherein the culture medium is kept under stirring during the reaction.

8. The process according to claim 1, wherein the cell density of the culture ranges from about 1.5 to about 20.

9. The process according to claim 1, wherein the incubation is kept between about 30 and about 50 hours.

10. The process according to claim 1, wherein the reaction temperature ranges from about 24 to about 36° C.

11. The process according to claim 1, wherein the reaction pH is between about pH 4.0 and pH 7.0.

12. A mixture of linalool derivatives obtained by a process as defined in claim 1.

13. A cosmetic composition comprising a mixture as defined in claim 12.
